(19) United States
(12) Patent Application Publication
(10) Pub. No.: US 2024/0041623 A1
(43) Pub. Date: Feb. 8, 2024

(54) STENT, IN PARTICULAR FOR TREATING CAROTID ARTERY DISEASES

(71) Applicant: Acandis GmbH, Pforzheim (DE)

(72) Inventors: Andreas Schüssler, Pfinztal (DE); Andrés Braschkat, Bretten (DE); Andreas Ding, Karlsruhe (DE)

(21) Appl. No.: 18/268,910

(22) PCT Filed: Dec. 21, 2021
(86) PCT No.: PCT/EP2021/086984

§ 371 Date: Jun. 21, 2023

(30) Foreign Application Priority Data
Dec. 22, 2020 (DE) 20 2020 107 453.9

(51) Int. Cl. A61F 2/90

(52) U.S. Cl. CPC A61F 2/90; A61F 2002/0081

(57) ABSTRACT

The disclosure relates to a stent, in particular for treating carotid artery diseases, including a tubular braided mesh made of wires which are each helically wound about a longitudinal axis of the braided mesh and cross over and under one another. In an idle state, the braided mesh has a proximal cylindrical portion and a distal cylindrical portion which are connected to one another via a transition portion, wherein the proximal cylindrical portion has a different cross-sectional diameter and a different porosity compared to the distal cylindrical portion.

The invention relates to a stent, in particular for the treatment of diseases of the carotid artery.

Stents are vessel supports which are usually formed from a tubular braided mesh which can expand from a small delivery diameter to a larger implantation diameter. The braided mesh is formed from wires, which wind helically about a longitudinal axis and thereby cross over and under each other. Commercially available stents have a largely cylindrical shape over their entire length.

Different sizes of stents are offered for the treatment of different blood vessels. When carrying out treatment in blood vessels, which have differing cross sectional diameters, however, such known stents frequently prove to be inadequate.

A disease which is normally treated with stents is the treatment of deposits in the carotid artery (arteria carotis). Deposits of this type are also known as soft plaques. Soft plaques often occur in vessels which branch off from the carotid artery, in particular the arteria carotis communis, so that upon implantation, the stent is positioned both in the larger main vessel (for example the arteria carotis communis) as well as in the branching blood vessel (for example the arteria carotis interna), which has a smaller cross sectional diameter.

In order to ensure that the stent is anchored well, the size of the stent which is to be inserted is selected on the basis of the larger vessel diameter. However, this means that in a distal section, i.e. in the smaller branching blood vessel, the stent is not expanded as much, whereupon the porosity of the stent in this section is increased compared with the porosity in the non-operational state. The surface area of the open meshes between the intersecting wires with respect to the surface area which is covered by the wires is therefore comparatively larger than in regions in which the stent is more expanded. Consequently, there is a risk that a large open surface area could be present in just that region of the blood vessel where a soft plaque is present, which should be pressed against the vessel wall by appropriately narrow meshes of the stent. This could have the result that particles becoming detached from the soft plaque could easily move through the meshes of the stent and therefore enter the bloodstream. As a consequence, embolisms could occur downstream.

In contrast, the porosity in a proximal region of the stent, which is positioned in a larger blood vessel and which therefore expands more, remains comparatively low. This is also undesirable, because blood vessels which branch from this region are no longer sufficiently supplied with blood. The lower porosity in the proximal section of the implanted stent therefore inhibits the flow of blood, whereas the comparatively large porosity in the distal section of the implanted stent can encourage detachment of embolism particles from a soft plaque.

In this respect, the objective of the invention is to provide a stent which has improved porosity properties when in the implanted state and is therefore well suited to is the treatment of diseases, in particular soft plaque, in the region of branched vessels.

In accordance with the invention, this objective is achieved by means of the subject matter of claim 1.

Thus, the invention is based on the concept of a stent, in particular for the treatment of diseases of the carotid artery, with a tubular braided mesh of wires which are respectively helically wound about a longitudinal axis of the braided mesh and which cross over and under each other. In a non-operational state, the braided mesh has a proximal cylindrical section and a distal cylindrical section which are connected together by a transitional section. In accordance with the invention, the proximal cylindrical section has a different cross sectional diameter and a different porosity to the distal cylindrical section.

Thus, the invention solves the problem in which different and unwanted regions of porosity are formed with a cylindrical stent in the implanted state through the provision of sections which can be constructively pre-set with an appropriate porosity, even in the non-operational state, i.e. in the state of the braided mesh in which no forces are applied to it. This means that the sections with different porosities are present in the desired form upon implantation of the stent in a blood vessel, in particular in the region of a branch of a vessel. Thus, for example, by appropriate adjustment of the porosity in the non-operational state, a distal section of the braided mesh, in particular the distal cylindrical section, has a smaller porosity than the proximal cylindrical section in the implanted state. Thus, the distal cylindrical section can efficiently shield a soft plaque in a branching blood vessel from the flow of blood, so that no particles can become detached. The risk of an embolism being initiated by insertion of the stent is therefore reduced. In contrast, the proximal cylindrical section which becomes positioned in a larger main blood vessel upon implantation has a larger porosity in the implanted state and therefore allows blood to flow through the meshes of the braided mesh in order, for example, to supply branching blood vessels with sufficient nutrients and oxygen.

In a preferred embodiment of the invention, the proximal cylindrical section has a higher porosity than the distal cylindrical section. This is the case for the non-operational state of the stent, in which no force is applied to the stent, i.e. it is not exposed to any external forces. The stent is expanded in the non-operational state. In this respect, the stent is preferably self-expandable. Specifically, the wires of the braided mesh may be formed from a shape memory material, in particular a nickel-titanium alloy, so that the stent expands autonomously to an expansion diameter which has been set by means of a heat treatment.

In general, unless otherwise indicated, in the present application, parameters of the braided mesh are described in the non-operational state. In the context of the application, however, a distinction is made between a porosity in the non-operational state and a porosity in the implanted state. Unless indicated otherwise, in general, reference is made to the porosity in the non-operational state. In each case of a reference to the porosity in the implanted state, this will be explicitly stated as such.

The proximal cylindrical section may have a larger cross sectional diameter than the distal cylindrical section. Particularly preferably, the proximal cylindrical section has a larger cross sectional diameter and a higher porosity than the distal cylindrical section.

During implantation of the stent in a blood vessel, the proximal cylindrical section preferably is positioned in a section of the blood vessel with a larger cross sectional diameter than the distal cylindrical section. In this regard, the stent size is chosen on the basis of the larger blood vessel section. Because the distal cylindrical section has a smaller cross sectional diameter, it fits the anatomy of the blood vessel to be treated better compared with purely cylindrical stents. Consequently, the distal cylindrical section can also expand sufficiently, so that in the distal cylindrical section, the porosity does not become larger than in the proximal cylindrical section.

In cylindrical stents up to now, exactly this effect can be observed, wherein a distally disposed stent section which, has not been completely expanded because of the position in a narrower blood vessel, attains a higher porosity. Specifically, it has therefore been shown that an insufficient expansion does not lead to a reduction in the porosity in the implanted state, but rather to an increase in the porosity in the implanted state. This is avoided by means of the reduction in the cross sectional diameter of the braided mesh in the distal cylindrical section compared to the proximal cylindrical section and the increased porosity in the proximal cylindrical section. Thus, in accordance with the invention, the porosity in the distal cylindrical section remains low in the implanted state so that, for example, a soft plaque can be properly covered by the distal cylindrical section of the stent.

In the non-operational state, the proximal cylindrical section may have a cross sectional diameter of at least 5 mm, in particular at least 8 mm, in particular at least 9 mm, in particular at least 10 mm. These sizes for the stents are particularly suitable for the treatment of soft plaque in the region of a bifurcation in the arteria carotis communis into the arteria carotis interna and the arteria carotis externa.

In a preferred embodiment of the invention, the distal cylindrical section has a different, in particular smaller, braiding angle than the proximal cylindrical section. The braiding angle influences the porosity in the individual sections of the braided mesh together with the cross sectional diameter. It has been shown that the porosity of the distal cylindrical section in the implanted state of the stent is advantageously influenced if, up to a cross sectional diameter of the distal cylindrical section of approximately 8 mm, the braiding angle in the distal cylindrical section is smaller than in the proximal cylindrical section. It is also possible, however, for the braiding angle in the distal cylindrical section and the braiding angle in the proximal cylindrical section to be the same. This is particularly appropriate for braided meshes which have a cross sectional diameter of more than 8 mm in the distal cylindrical section. Since with such larger cross sectional diameters, the individual wires of the braided mesh, which run parallel to each other, are separated from each other by a larger distance, the porosity of the braided mesh in the implanted state rises in that region even when the braiding angle remains the same. This knowledge regarding the relationship between cross sectional diameter and braiding angle is particularly advantageous for the production of stents in different sizes, which fulfil the function of obtaining good coverage of soft plaque in branching blood vessels and at the same time ensure good perfusion of the main blood vessel, in which the proximal cylindrical section of the stents is positioned.

In preferred embodiments of the invention, the braiding angle in the distal cylindrical section is between 60° and 75°, in particular between 63° and 72°, in particular between 63° and 67° or between 68° and 72°, preferably 65° or 70°. For braided meshes for which the cross sectional diameter of the distal cylindrical section is less than 8 mm, a braiding angle in the distal cylindrical section of between 60° and 70°, in particular between 63° and 67°, advantageously 65°, is advantageous.

For braided meshes with a cross sectional diameter of more than 8 mm in the distal cylindrical section, the braiding angle in the distal cylindrical section is preferably between 68° and 72°, preferably 70°. In the proximal cylindrical section, the braiding angle is preferably between 65° and 75°, in particular between 68° and 72°, in particular 70°. This may be the case for all sizes of stents, i.e. all of the different cross sectional diameters for the proximal cylindrical section which are offered.

In some variations of the invention, the distal cylindrical section may comprise a distal longitudinal end of the braided mesh in which the wires form end loops. The wires which form the braided mesh may therefore be helically wound about a longitudinal axis, be bent round at the distal longitudinal end and then be helically wound about the longitudinal axis in the opposite direction. As an alternative, end loops may be formed at the proximal longitudinal end of the braided mesh. In this manner, end loops are formed at the distal longitudinal end. The end loops have the advantage that during implantation, the vessel walls are barely affected if they are contacted, and therefore have an atraumatic effect.

The distal longitudinal end may also be conically flared. In particular, the end loops may be curved radially outwardly, so that the distal longitudinal end can anchor the braided mesh in a blood vessel to better effect. Despite the slightly conical profile of the distal longitudinal end, in the context of this application, the distal longitudinal end is considered to be part of the distal cylindrical section.

The transitional section which connects the proximal cylindrical section and the distal cylindrical section together may be conical in shape. It is also possible, in fact, for the transitional section to be convex or concave. However, the conical shape is preferred, not only from the point of view of manufacturing technology, but also because of the way the transitional section is seated on the vessel walls; it is particularly uniform in the case of the conical shape.

Specifically, the transitional section may have a cone angle of between 5° and in particular between 7° and 15°, preferably 10°. It has been shown that with a cone angle of this type and appropriate ratios of the diameters between the proximal cylindrical section and the distal cylindrical section, the flow of blood through the braided mesh, in particular in a branching blood vessel, is also positively influenced. This is particularly the case when, as is further preferable, the braiding angle within the transitional section is between 60° and 75°, preferably between 63° and 73°.

The braided mesh may have between 12 and 36 wires, in particular between 18 and 30 wires, preferably 24 wires. In a variation of the stent in which the braided mesh forms end loops at the distal longitudinal end, a corresponding number of end loops is provided, for example 24 end loops when 24 wires are used. Because the 24 wires are bent round in order to form the end loops, then consequently, there will be 48 wire ends at the proximal longitudinal end of the braided mesh.

The wires may have a wire diameter of between 50 μm and 120 μm, in particular between 60 μm and 100 μm, in particular between 70 μm and 90 μm, preferably 80 μm. These wire diameters are particularly suitable for obtaining good anchoring of the stent in the arteria carotis. The wire diameter has a significant influence on the radial force with which the braided mesh presses against the vessel wall. Particular materials for use as the wires are nickel-titanium alloys, in particular nitinol. It is also possible for the wires or for at least some of the wires to contain a composite material, wherein a core of the wires is formed by a radiopaque material, for example platinum or platinum-iridium, whereas a shell material which sheaths the core material is formed by a nickel-titanium alloy. Preferably, in all cases a material is selected which endows the stent with self-expandable properties. In other words, the braided mesh may be self-expandable.

The invention will now be described in more detail with the aid of an exemplary embodiment and with reference to the accompanying schematic drawings, in which:

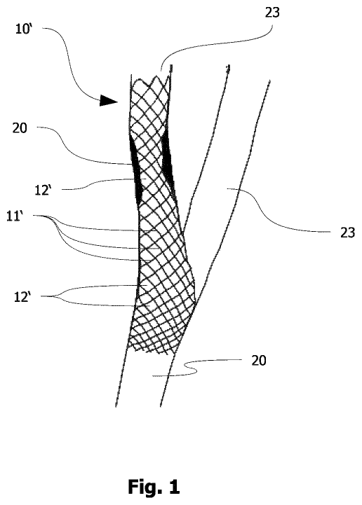

FIG. 1 shows a side view of a conventional cylindrical stent in the implanted state in the region of the bifurcation of the arteria carotis communis into the arteria carotis interna and the arteria carotis externa;

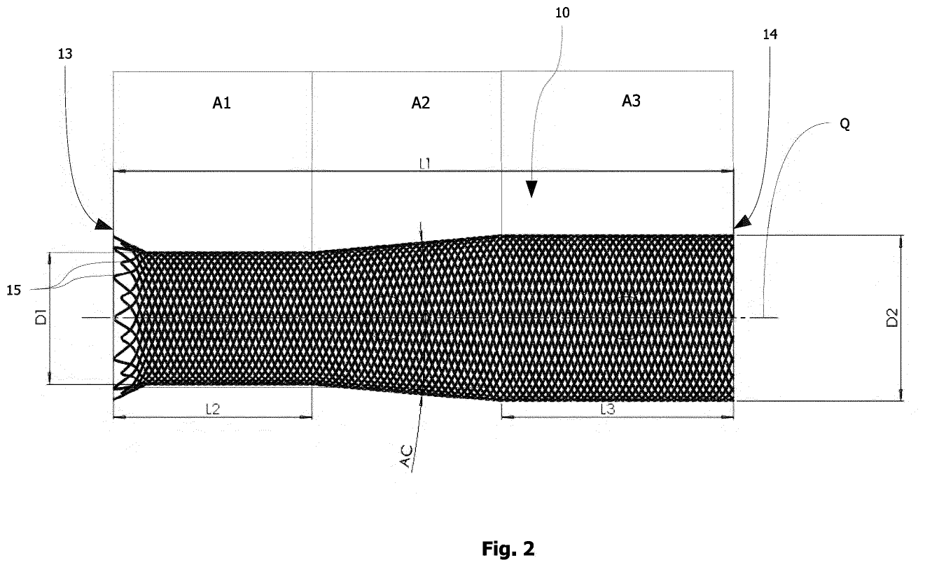

FIG. 2 shows a side view of a stent in accordance with the invention in a preferred exemplary embodiment; and

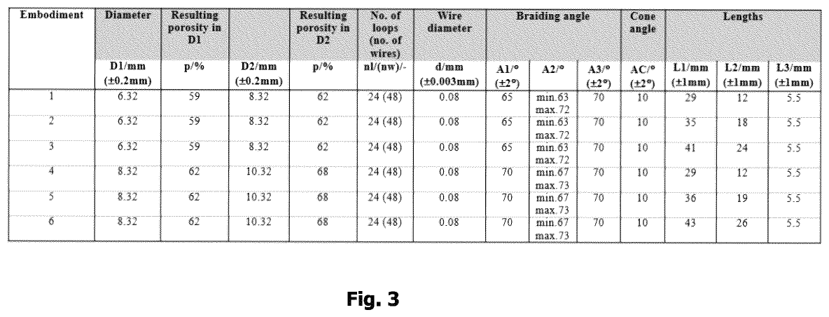

FIG. 3 shows a tabular overview of parameters for different exemplary embodiments of the stent in accordance with FIG. 2.

FIG. 1 illustrates the problem which underlies the invention described here. Thus, FIG. 1 shows the bifurcation of the arteria carotis communis 21 which divides into the arteria carotis interna 22 and the arteria carotis externa 23. A soft plaque 20 is formed in the arteria carotis interna 22. In order to prevent the soft plaque 20 from further inhibiting the flow of blood through the arteria carotis interna 22, a conventional cylindrical stent 10′ is inserted. The task of the stent 10′ is to push the soft plaque 20 onto the vessel wall of the arteria carotis interna 22 and therefore to dilate the arteria carotis interna 22 in this region in order to re-establish a sufficient flow of blood to supply downstream regions of tissue with nutrients and oxygen. Because the soft plaque 20 is located close to the bifurcation, it is necessary to implant the stent 10′ in a manner such that it is positioned with a proximal section in the arteria carotis communis 21 and with a distal section in the arteria carotis interna 22.

The stent is braided from wires 11′ which respectively delimit meshes 12′. Because of the difference in cross sectional diameters which arises during implantation of the stent 10′, the position of the wires 11 with respect to each other also changes, which has an effect on the size of the meshes 12′ in the implanted state. As a result, the porosity in the implanted state is also influenced.

In FIG. 1, it can be seen that the meshes 12′, for example, have a larger mesh size in the region of the soft plaque 20 than in the region of the arteria carotis communis 21. Because the arteria carotis interna 22 has a smaller cross sectional diameter, the stent 10′ in this region expands less, which means that in the implanted state, the surface area of the mesh openings 12 in the distal section of the stent is larger overall than in the proximal section in which the stent 10′ can deploy further in the arteria carotis communis 21. The larger meshes 12′ in the region of the soft plaque 20 cannot hold back particles of the soft plaque 20 to a sufficient extent. The mesh size in this region is too large to be able to safely retain all of the soft plaque particles 20. Thus, soft plaque particles 20 can detach and be washed by the flow of blood in the arteria carotis interna 22 in the direction of downstream, smaller blood vessels and lead to an occlusion therein.

In the proximal portion of the conventional stent 10′, however, it expands further in the region of the branch of the arteria carotis externa 23, which as a result, makes the mesh size of the meshes 12′ in this region smaller. Thus, in the implanted state, a porosity is set which is smaller than in the proximal portion of the stent 10′. Thus, the flow of blood into the arteria carotis externa 23 is more severely inhibited than desired and this can lead to secondary disease.

The invention prevents these effects from happening by providing a stent 10 which has different cross sectional diameters and sections with different porosities in the non-operational state, so that in the implanted state, the desired porosity properties can be obtained. A stent of this type is shown by way of example in FIG. 2.

The stent 10 of FIG. 2 has a braided mesh of wires 11 which respectively form meshes. In this regard, the wires 11 are helically wound about a longitudinal axis Q and cross over and under each other in a regular manner. The wires 11 are helically wound about the longitudinal axis Q starting from a proximal longitudinal end 14 up to a distal longitudinal end 13. At the distal longitudinal end 13, the wires 11 are each bent round and form end loops 15. The bent wires are then wound helically in the opposite direction about the longitudinal end axis Q up to the proximal longitudinal end 14. Thus, open wire ends are present at the proximal longitudinal end 14, wherein the number of the wire ends corresponds to twice the number of the end loops 15.

The stent of FIG. 2 is divided into three sections A1, A2, A3. A proximal cylindrical section A3 is formed, starting from the proximal longitudinal end 14. A transitional section A2 directly adjoins the proximal cylindrical section A3. The transitional section A2 transitions into a distal cylindrical section A1.

The distal cylindrical section A1 comprises the distal longitudinal end 13 with the end loops 15. FIG. 2 also shows that the end loops 15 are curved radially outwardly, and so a conically flared distal longitudinal end 13 is formed. In the context of the present application, the conically flared distal longitudinal end 13 is considered to be part of the distal cylindrical section A1.

As can be seen in FIG. 2, the distal cylindrical section A1 has a cross sectional diameter D1 which is smaller than the cross sectional diameter D2 of the proximal cylindrical section A3. The transitional section A2 connects the proximal cylindrical section A3 to the distal cylindrical section A1 and is essentially conical in form. In other words, the transitional section A2 tapers from the proximal cylindrical section A3 to the distal cylindrical section A1. In this regard, the transitional section A2 has a cone angle AC which f results from the length of the transitional section A2 and the difference between the cross sectional diameter of the proximal cylindrical section A3 and the cross sectional diameter of the distal cylindrical section A1.

FIG. 2 shows the stent 10 in the non-operational state, i.e. without any force being applied. In the implanted state, an external force acts on the stent 10 which results in the braided mesh of the stent being pushed against the vessel wall with a radial force which preferably results from the self-expanding properties of the braided mesh. In order to obtain good anchoring in the blood vessel, in order to treat the blood vessel, a stent is preferably selected which has a cross sectional diameter that is somewhat larger (usually ca. 20% larger) than the largest cross sectional diameter of the blood vessel to be treated (oversizing). Thus, the stent 10 is dimensioned so that in the implanted state, there is an expansion reserve which acts to produce a radial force which permanently pushes the stent 10 against the blood vessel wall. This means that in the implanted state, the braided mesh of the stent 10 takes up a smaller cross sectional diameter than in the non-operational state.

Consequently, in the implanted state, the positions of the wires 11 with respect to each other change due to the resulting compression and extension of the stent 10. This in turn influences the size of the meshes 12. The invention takes advantage of this mechanism in order to provide the desired porosity, in particular for the distal cylindrical section A1.

Particularly preferably in the case of the stent 10 in accordance with FIG. 2, a higher porosity is set up in the proximal cylindrical section A3 than in the distal cylindrical section A1. When the stent in accordance with FIG. 2 is inserted in the region of the bifurcation of the arteria carotis communis 21, as was the case for the conventional stent 10′ in FIG. 1, the distal cylindrical section A1 is positioned in the region of the soft plaque 20. Because the distal cylindrical section A1 has a smaller porosity than the proximal cylindrical section A3, the soft plaque 20 is efficiently retained. In this regard, the stent 10 takes advantage of two effects. On the one hand, in the non-operational state, the porosity in the distal cylindrical section A1 is already small. On the other hand, the cross sectional diameter of the distal cylindrical section A1 is also smaller in the non-operational state, so that in the implanted state, the distal cylindrical section A1 is less severely compressed than is the case with the purely cylindrical stent 10′ of FIG. 1. In this manner, the distal cylindrical section A1 retains almost the same porosity when in the implanted state as it has in the non-operational state.

The porosity of the distal cylindrical section A1 is preferably set so that particles which could lead to the formation of occlusions in downstream blood vessels cannot penetrate through the narrow meshes in the distal cylindrical section A1. The comparatively higher porosity in the proximal cylindrical section A3, which is disposed in the arteria carotis communis 21 in the implanted state, for example, and/or of the transitional section A2, which spans the arteria carotis externa 23, has the opposite effect, in that blood can flow through the meshes so that a sufficient supply to the arteria carotis externa 23 is ensured.

It has been shown that specific parameters for implementing the function of the stent in accordance with FIG. 2 are particularly advantageous. The table in FIG. 3 shows six preferred exemplary embodiments of a stent 10 in accordance with the invention. For all of the exemplary embodiments cited in the table, the stent 10 is a braided mesh produced from 24 wires with a wire diameter of 80 μm. The cone angle AC in the transitional section A2 is advantageously 10° in all of the exemplary embodiments of FIG. 3. In addition, the proximal cylindrical section A3 has a braiding angle of 70° in all of the exemplary embodiments of FIG. 3. The tolerances given in the table preferably apply to all of the parameters which have been described.

Regarding the number of wires given in the table, it should be noted that these essentially describe the number of open wire ends at the proximal longitudinal end 14. Because each wire 11 is bent round at the distal longitudinal end 13 in order to form an end loop 15, the number of wires in fact corresponds to the number of loops. However, the table of FIG. 3 gives only the number of wire ends or the number of wires over a circumferential segment of the stent, without considering the fact that the wires are bent round at the end loops 15.

The following applies for all of the embodiments and exemplary embodiments of the invention:

The proximal cylindrical section A3 has a different porosity to the distal cylindrical section A1. In general, the porosity can be specifically set in a manner such that it is dependent on the stent diameter in one of the sections A1, A3. Other parameters which could influence the porosity are the wire diameter, the number of wires of the braided mesh and the braiding angle. In particular, the porosity Por, can be set in at least one of the cylindrical sections A1, A3 using the following interdependency with respect to the cited parameters:

$$Por = 1 - \frac{\frac{d n}{cos α} - \frac{2 {d}^{2}}{n π D tan α sin 2 α}}{π D}$$

in which “d” designates the diameter of the wire 11, “n” designates the number of wires, “a” designates the braiding angle and “D” designates the external diameter of the stent 10.

In order to obtain the result as a percentage, the value for the porosity Por, has to be multiplied by 100:

Por(%)=Por×100

The porosity describes the ratio of the projected wire surface area (minus the surface area of the intersections) to the total curved surface area of the stent 10.

Particularly preferably, the porosity of the proximal cylindrical section A3 differs from the porosity of the distal cylindrical section A1 by at least 2%, in particular at least 2.5%, in particular at least 3%. The difference may result in a higher porosity or a lower porosity of the proximal cylindrical section A3 compared with the distal cylindrical section A1. The porosity can also be adjusted by twisting the wires 11, 11′ along the longitudinal axis in pairs.

### LIST OF REFERENCE NUMERALS

10′, 10 stent
11′, 11 wire
12′, 12 mesh
13 distal longitudinal end
14 proximal longitudinal end
15 end loop
20 soft plaque
21 arteria carotis communis
22 arteria carotis interna
23 arteria carotis externa
A1 distal cylindrical section
A2 transitional section
A3 proximal cylindrical section
Q longitudinal axis

What is claimed is:

1.-13. (canceled)

14. A stent comprising:
a tubular braided mesh of wires, each wire respectively helically wound about a longitudinal axis of the braided mesh and arranged to cross over and under each other wire,
wherein in a non-operational state, the braided mesh has a proximal cylindrical section and a distal cylindrical section connected by a transitional section, and
wherein the proximal cylindrical section has a cross sectional diameter and a porosity that are different than that of the distal cylindrical section.

15. The stent as claimed in claim 14, wherein the proximal cylindrical section has a higher porosity than the distal cylindrical section.

16. The stent as claimed in claim 14, wherein the proximal cylindrical section has a larger cross sectional diameter than that of the distal cylindrical section.

17. The stent as claimed in claim 14, wherein in the non-operational state, the cross sectional diameter of the proximal cylindrical section is at least 5 mm.

18. The stent as claimed in claim 14, wherein the distal cylindrical section has a braiding angle that is different than that of the proximal cylindrical section.

19. The stent as claimed in claim 18, wherein the braiding angle (i) in the distal cylindrical section is between 60° and 75° and (ii) in the proximal cylindrical section is between 65° and 75°.

20. The stent as claimed in claim 14, wherein the distal cylindrical section comprises a distal longitudinal end of the braided mesh wherein the wires form end loops.

21. The stent as claimed in claim 20, wherein the distal longitudinal end is conically flared.

22. The stent as claimed in claim 14, wherein the transitional section is conical in shape.

23. The stent as claimed in claim 22, wherein the transitional section has a cone angle of between 5° and 20°.

24. The stent as claimed in claim 14, wherein the braided mesh has between 12 and 36 wires.

25. The stent as claimed in claim 14, wherein the wires have a wire diameter of between 50 μm and 120 μm.

26. The stent as claimed in claim 14, wherein the porosity of the proximal cylindrical section differs from the porosity of the distal cylindrical section by at least 2% and wherein the porosity of the proximal cylindrical section is one of larger or smaller than the porosity of the distal cylindrical section.

27. A stent comprising:
a tubular braided mesh of wires, each wire respectively helically wound about a longitudinal axis of the braided mesh and arranged to cross over and under each other wire,
wherein in a non-operational state, the braided mesh has a proximal cylindrical section and a distal cylindrical section connected by a transitional section,
wherein the proximal cylindrical section has a cross sectional diameter and a porosity that are different than that of the distal cylindrical section,
wherein the proximal cylindrical section has a higher porosity and a larger cross sectional diameter than the distal cylindrical section, and
wherein the distal cylindrical section has a braiding angle that is different than the proximal cylindrical section.

28. The stent as claimed in claim 27, wherein the distal cylindrical section comprises a distal longitudinal end of the braided mesh wherein the wires form end loops.

29. The stent as claimed in claim 28, wherein the distal longitudinal end is conically flared.

30. The stent as claimed in claim 27, wherein the transitional section is conical in shape.

31. The stent as claimed in claim 30, wherein the transitional section has a cone angle of between 5° and 20°.

32. The stent as claimed in claim 27, wherein the porosity of the proximal cylindrical section differs from the porosity of the distal cylindrical section and wherein the porosity of the proximal cylindrical section is one of larger or smaller than the porosity of the distal cylindrical section.

33. The stent as claimed in claim 27, wherein the braided mesh has between 18 and 30 wires.
